(19) United States
(12) Patent Application Publication
(10) Pub. No.: US 2002/0198176 A1
(43) Pub. Date: Dec. 26, 2002

(54) Treatment of psoriasis with matrix metalloproteinase inhibitors

(76) Inventor: Raul Fleischmajer, Barnegat Light, NJ (US)

Correspondence Address: BAKER & BOTTS, 30 ROCKEFELLER PLAZA, NEW YORK, NY, 10112

(21) Appl. No.: 10/207,593

(22) Filed: Jul. 29, 2002

Related U.S. Application Data
(63) Continuation of application No. 09/794,660, filed on Feb. 27, 2001, now abandoned.
(60) Provisional application No. 60/186,431, filed on Mar. 2, 2000.

(51) Int. Cl. A61K 31/66; A61K 31/65; A61K 31/19

(52) U.S. Cl. 514/75; 514/557; 514/152; 514/575

(57) ABSTRACT

The present invention relates to methods of treating psoriasis by inhibiting one or more matrix metalloproteinase enzymes (“MMPs”). It is based, at least in part, on the discovery that the expression patterns of certain MMPs and related molecules are altered in patients suffering from psoriasis, relative to normal subjects. Certain expression patterns are altered even in unaffected skin of psoriasis-afflicted patients, although aberrancies are more pronounced in psoriatic lesions. In various non-limiting embodiments, the present invention provides for methods of treating psoriasis, including preventing the development of new psoriatic lesions, comprising administering, to subjects in need of such treatment, effective concentrations of compounds which inhibit the enzymatic activity of one or more MMP. Suitable inhibitors include tetracycline and its derivatives and various hydroxymate, carboxylic acid, and phosphonic acid derivatives. Therapy may comprise systemic and/or local administration of inhibitor. In additional embodiments, the present invention provides for methods of diagnosing MMP inhibitor responsive skin lesions, for evaluating the level of disease activity in a subject, and for transgenic animal and tissue culture models of psoriasis.

### SPECIFICATION

This application claims priority to U.S. Provisional Patent Application No. 60/186,431, filed Mar. 2, 2000.

### 1. INTRODUCTION

The present invention relates to methods of treating psoriasis by inhibiting matrix metalloproteinase enzyme(s). It is based, at least in part, on the discovery that the expression of certain matrix metalloproteinase enzyme(s) is increased in the epidermis of patients suffering from psoriasis, and particularly increased in psoriatic skin lesions.

### 2. BACKGROUND OF THE INVENTION

### 2.1. Psoriaris

Psoriasis is a chronic skin disease characterized by red scaly patches that usually affect the scalp, elbows and knees, although any part of the skin may be involved. At the cellular level, psoriasis is a benign proliferative disease of keratinocytes of unknown etiology. It has been estimated that psoriasis affects about 2 percent of the population in Western countries, 0.1 to 0.3 percent in the Far East and is rather rare in persons of the black race (Krueger et al., 1984, J. Am. Acad. Dermatol. 11:937-947; Yui-Yip, 1984, J. Am. Acad. Dermatol. 10:965-968). Although the disease appears to be inherited, its mode of transmission is not known and more than one genetic locus may be involved (Henseler, 1997, J. Am. Acad. Dermatol. 37:S1-11). Furthermore, the disease can be triggered or exacerbated by external factors such as trauma, infection and drugs.

Histologically, the skin pathology is characterized by acanthosis, thickening of the epidermis, angiogenesis of superficial blood vessels and an inflammatory response. It is not known whether the primary alteration in psoriasis resides in the keratinocytes or is the result of an autoimmune process. With regard to the latter alternative, there is evidence that an epidermal antigen triggers the appearance of neutrophils, macrophages and activated T-lymphocytes, mostly CD8+T cells (Chang et al., 1994, Proc. Natl. Acad. Sci. (U.S.A.) 21:9283-9286). This immune response results in the release of various cytokines (IL-1, IL-6, IL-8, TNF-α) which may be responsible for keratinocyte proliferation and angiogenesis (Menssen et al., 1995, J. Immunol. 155:4078-4083). Another school of thought suggests that cell adhesion of keratinocytes may be altered in psoriasis and that these changes may involve cell-cell and cellmatrix interactions; studies have shown decreased adhesiveness between keratinocytes (Orfanos et al., 1973, Arch. Dermatol. 107:38-46) and alterations of the basement membrane at the epidermal-dermal interface (Mondello et al., 1996, Arch. Dermatol. Res. 288:527-531). In addition, redistribution of α₃β₁ and α₆β₄ integrins from basal to suprabasal keratinocyte layers was noted in both uninvolved and involved skin (Hertle et al., 1992, J. Clin. Invest. 89:1882-1901; Pellegrini et al., 1992, J. Clin. Invest. 89:1783-1795). It has also been shown that transgenic mice expressing integrins in the suprabasal layer of the epidermis developed a phenotype that closely resembled psoriasis (Carroll et al., 1995, Cell 83:957-968).

Current treatments for psoriasis attempt to control keratinocyte proliferation, promote differentiation, and reduce the associated inflammatory reaction. Systemic therapy involves the use of cytotoxic drugs (e.g., methotrexate, cyclosporin) or retinoids. Topical therapy consists of corticosteroids, calcipotriene (an analog of Vitamin D) and acetylenic retinoid which is rapidly converted into tazarotenic acid. In addition, ultraviolet B or ultraviolet A radiation combined with oral methoxypsoralen for photosensitization has also been used in the treatment of psoriasis. Although the above-listed treatments may be of benefit to some patients, others may fail to respond to conventional therapies and thus psoriasis becomes a difficult therapeutic challenge to the clinician. In addition, the chronic use of immunosuppressive drugs, retinoids, or ultraviolet light may result in undesirable side effects, including depression of immunity, liver disease, fetal abnormalities and skin cancer.

### 2.2. Matrix Metalloproteinases

Matrix metalloproteinases (“MMPs”) are zinc-dependent endopeptidases involved in the remodeling of the extracellular matrix (“ECM”). MMPs play an important role in morphogenesis, angiogenesis, wound healing, and in certain disorders such as rheumatoid arthritis, tumor invasion and metastasis (Birkedal-Hansen, 1995, Curr. Opin. Cell Biol. 7:728-735). Five subfamilies of MMPs have been recognized: collagenases, gelatinases, stromelysins, matrilysins, and membrane-type MMPs. These enzymes contain propeptide, catalytic and hemopexin (except for matrilysins) domains and are involved in the degradation of collagens, proteoglycans and various glycoproteins (Id.). MMPs are secreted as inactive zymogens (pro-MMPs) and their activation (to “a-MMPs”) is a prerequisite for function. Stimulation or repression of most pro-MMP synthesis is regulated at the transcriptional level by growth factors and cytokines. In vivo activation of pro-MMPs involves the removal of the propeptide by serine proteases (e.g., trypsin, plasmin, etc.; Id.).

Furthermore, post-translational regulation of MMP activity is controlled by tissue inhibitors of MMPs (“TIMPs”), four of which have been characterized and designated as TIMP-1, TIMP-2, TIMP-3, and TIMP-4 (Gomez et al., 1997, Eur. J. Cell. Biol. 74:111-122). MMP-2 or gelatinase A (72 kDa type IV collagenase) and MMP-9 or gelatinase B (92 kDa type IV collagenase) degrade basement membrane (“BM”) and have been incriminated in the mechanism of tumor invasion and metastasis. A distinctive structural feature of both MMP-2 and MMP-9 is the presence, in their catalytic domains, of three tandem repeats of fibronectin type III modules that enable these pro-enzymes and their active forms to bind to gelatin (Collier et al., 1992, J. Biol. Chem. 267:6776-6781). MMP-2 binds specifically to TIMP-2 while MMP-9 binds to TIMP-1 (Goldberg et al., 1989, Proc. Natl. Acad. Sci. (U.S.A.) 86:8207-8211).

MMP-2 has several unique structural and functional characteristics that distinguish it from all other MMPs. MMP-2 is constitutively expressed in many cells and has a ubiquitous tissue distribution, its promoter lacks a conventional TATA box, AP-1 and PEA-3 site enhancers, and it is not stimulated by serine proteases or tissue plasminogen activator (TPA) (Birkedal-Hansen, 1995, Curr. Opin. Cell Biol. 7:728-735). In addition, MMP-2 responds poorly to growth factors or cytokines, although it can be moderately stimulated by TGF-β1 (Salo et al., 1991, J. Biol. Chem. 266:11436-11441; Overall et al., 1991, J. Biol. Chem. 266:14064-14071). Pro-MMP-2 is activated at the cell surface by a cell membrane MMP known as MT1-MMP (Sato et al., 1994, Nature 370:61-65; Okada et al., 1990, Eur. J. Biochem. 194:721-730; Sato et al., 1996, J. Biochem. 119:209-215).

It has been shown that tetracyclines, besides acting as antibiotics, can inhibit MMPs (Golub et al., 1991, Crit. Rev. Oral Biol. Med. 2:297-322). Chemical modifications of tetracyclines may eliminate antimicrobial properties of the modified compounds without affecting the ability to inhibit MMPs. Doxycycline, a synthetic tetracycline antibiotic, has been shown to inhibit MMPs (Golub et al., 1983, J. Periodent. Res. 18:516-566). It is also noteworthy that doxycycline and other chemically-modified tetracyclines can inhibit MMP-2 mRNA production in cultured keratinocytes (Uitto et al., 1994, Ann. N.Y. Acad. Sci. 732:140-151) and can inhibit keratinocyte migration (Makela et al., 1998, Adv. Dent. Res. 12:131-135). Currently, clinical trials are being conducted with various tetracyclines for the treatment or prevention of abdominal aortic aneurysms, prostate cancer, periodontal disease and osteoporosis (Greenwald et al., 1999, Ann. New York Acad. Sci. 878:1-761).

### 3. SUMMARY OF THE INVENTION

The present invention relates to methods of treating psoriasis by inhibiting one or more matrix metalloproteinase enzymes (“MMPs”). It is based, at least in part, on the discovery that the expression patterns of certain MMPs and related molecules are altered in patients suffering from psoriasis, relative to normal subjects. Certain expression patterns are altered even in unaffected skin of psoriasis-afflicted patients, although aberrancies are more pronounced in psoriatic lesions.

In various non-limiting embodiments, the present invention provides for methods of treating psoriasis, including preventing the development of new psoriatic lesions, comprising administering, to subjects in need of such treatment, effective concentrations of compounds which inhibit the enzymatic activity of one or more MMP. Suitable inhibitors include tetracycline and its derivatives and various hydroxymate, carboxylic acid, and phosphonic acid derivatives. Therapy may comprise systemic and/or local administration of inhibitor.

In additional embodiments, the present invention provides for methods of diagnosing MMP inhibitor responsive skin lesions, and for determining the degree of activity of disease based on serum or plasma enzyme levels. The present invention also provides for transgenic animal and tissue culture models of psoriasis.

Subsequent to the filing of the provisional application on which this application is based, the subject matter of the invention was published, in part, in Fleischmajer et al., November 2000, J. Invest. Dermatol. 115(5):771-777.

### 4. DESCRIPTION OF THE FIGURES

FIGS. 1A-D. Transmission electron microscopy of psoriatic skin. Psoriasis-uninvolved (Ps-U) skin reveals a normal epidermis (A) and a normal epidermal-dermal interphase (B). Psoriasis-involved (Ps-I) skin shows widening of keratinocyte intercellular spaces, a reduction in desmosomes and narrow, elongated intercellular bridges (arrow) suggesting an alteration in cell-cell adhesion (C). Basal keratinocytes reveal numerous vesicles (V) and gaps (arrow heads) of the lamina densa (D). Bar=100 nm (B and D). Bar=1.5 μm (A and C).

FIGS. 2A-D. Immunocytochemistry microscopic analysis of basement membrane using mAbs against collagen IV and laminin chains. Normal skin control (N) shows linear staining of the basement membrane. (A). Psoriasis-involved (Ps-I) skin shows gaps, reduced staining intensity (B-D) and excessive folding (C) of the basement membrane (Arrows). Magnification=×460.

FIGS. 3A-D. Immunocytochemistry analysis by confocal laser microscopy using mAbs against MMP-2 and MMP-9. Psoriasis-uninvolved (Ps-U) skin shows MMP-2 in the cytoplasm of several suprabasal keratinocytes; arrowhead points to the basal cell layer (A). Psoriasis-involved (Ps-I) skin shows intense staining for MMP-2 in most keratinocytes of the suprabasal layer (B). Note that MMP-9 is absent in Ps-I (C). Normal control skin was negative for MMP-2 (D). Magnification=×2300.

FIGS. 4A-D. Immunocytochemistry analysis using mAb against TIMP-2. Psoriasis-uninvolved (Ps-U) skin shows TIMP-2 in basal keratinocytes, mostly at the epidermal-dermal interface (A). Psoriasis-involved (Ps-I) skin shows TIMP-2, in a distinct pericellular pattern in suprabasal keratinocytes. Arrowhead points to the basal cell layer (B). High magnification by confocal laser microscopy shows TIMP-2 at the cell surface (C). Non-reactive control serum (D). A, B and D, magnification=×460; C, magnification=×2300.

FIG. 5. Gelatin zymography for MMP-2 and MMP-9. Normal control skin (N) only expressed pro-MMP-2. Psoriasis-uninvolved (Ps-U) and psoriasis-involved (Ps-I) skin shows pro-MMP-2 and a-MMP-2 (active form) in 3 patients. Note that pro-MMP-9 was only expressed in Ps-I skin. CM=Conditioned medium from NIH-3T3 cell cultures as positive control for MMP-2.

FIGS. 6A-B. Western blots for MMP-2 (A) and TIMP-2 (B). Note that patients 1 and 2 express pro-MMP-2 and a-MMP-2 in uninvolved (Ps-U) and involved (Ps-I) skin. However, note the predominance of a-MMP-2. Normal control skin (N) revealed none or only pro-MMP-2. TIMP-2 expression is increased in Ps-U and Ps-I skin, as compared to control

FIGS. 7A-B. Western blots for MT1-MMP. Note increased expression of MT1-MMP in psoriasis-involved (Ps-I) skin, as compared to psoriasis-uninvolved (Ps-U) and normal control skin (N), in Patient 1 (A) and Patient 2 (B). C=culture medium from human endothelial cells of dermal origin.

FIGS. 8A-D. In situ hybridization for MMP-2 mRNA. Note marked expression of MMP-2 mRNA in psoriasis-uninvolved (Ps-U) and psoriasis-involved (Ps-I) skin (A and B). Normal control skin revealed weak signals (D). The sense mRNA probe was negative (C). ×230.

### 5. DETAILED DESCRIPTION OF THE INVENTION

The present invention relates to methods of treating psoriasis comprising administering, to a subject in need of such treatment, a therapeutically effective amount of an inhibitor of a matrix metalloproteinase enzyme (“MMP”).

The term “treating” as defined herein refers to reducing the number or size or thickness of psoriatic lesions in a patient already suffering from psoriasis and/or reducing the discomfort associated with said lesions. The tern also refers to inhibiting the growth of preexisting lesions and to preventing the development of new lesions in a patient already suffering from psoriasis or a patient predisposed to develop psoriasis. Thus, reducing the number or size or thickness of psoriatic lesions, reducing the discomfort associated with psoriatic lesions, inhibiting the growth of preexisting lesions, or inhibiting the growth or development of new lesions constitutes “treating” psoriasis according to the invention.

MMP inhibitors according to the invention inhibit the enzymatic activity of one or more members of the MMP family, and preferably inhibit MMP-2 and/or MMP-9. The ability of a compound to inhibit an MMP may be determined, for example, using a standard assay in which the ability of an MMP to act upon its substrate is compared in the presence and in the absence of a putative inhibitor. Suitable substrates include, but are not limited to, gelatin, which may optionally be labeled, for example, radiolabeled (see, for example, Makela et al., 1998, Adv. Dent. Res. 12:131-135). Preferably, but not by way of limitation, a MMP inhibitor used according to the invention decreases the activity of an MMP, for instance MMP-2 and/or MMP-9, by at least the same extent as the decrease in activity caused by the same molar concentration of tetracycline. A review of MMP inhibitors is presented in Part II of “Inhibition of Matrix Metalloproteinases: Therapeutic Applications”, (Greenwald et al., eds, 1999, Annals N.Y. Acad. Sci. 878: 40-107, and entitled “Optimizing the Design of Inhibitors”.

MMP inhibitors which may be used to treat or prevent psoriasis according to the invention include, but are not limited to, tetracycline and its derivatives, including but not limited to: natural tetracyclines, such as chlortetracycline, oxytetracycline, and tetracycline; semi-synthetic tetracyclines such as minocycline, doxycycline, and methacycline; and chemically modified tetracyclines (“CMTs”)such as CMT-1 (4-dedimethylamino-tetracycline), CMT-2 (tetracycline-nitrile), CMT-3 (6-demethyl-6-deoxy-4-dedimethylaminotetracycline); CMT-4 (7-chloro-4-dedimethylaminotetracycline); CMT-5 (tetracyclinepyrazole); CMT-6 (4-dedimethylamino-4-hydroxytetracycline); CMT-7 (12-alpha-deoxy-4-dedimethylaminotetracycline); and CMT-8 (6 alpha deoxy-5 hydroxy-4-dedimethylaminotetracycline).

Other MMP inhibitors which may be used according to the invention include but are not limited to hydroxamic acid (Conhoh); synthetic MMP inhibitors (which achieve inhibition through zinc-binding groups) including hydroxamate compounds, carboxylate compounds, aminocarboxylate compounds, sulphhydryl compounds, phosphoric acid derivatives, mercaptoalcohols, and mercaptoketones (Beckett et al., 1996, Drug Dev. Today 1:16-26; Morphy et al., 1994, Bioorg. Med. Chem. Lett. 4:2747-2752; Skotnicki et al., 1999, Ann. N.Y. Acad. Sci. 878:61-72), and the specific compounds Batimastat (BB-94; British Biotechnol.), Marimastat (BB-2516; British Biotechnol. ), Ilomastat (GM6001; Glycomed), CT-1746 (Celltech), AG-3340 (Agouron), BAY 12-9566 (Bayer), CGS27023A (Novartis), D-5419 (Chiroscience), R0 32-3555 (Roche), G1168 (Glaxo Wellcome), G 1173 (Glaxo Wellcome) and CDP-845 (Celltech); and natural products that carry hydroxamic acid, including BE 16627B (Banyis; Naito et al., 1993, Agents & Actions 39:182-186), and Matlystatin B (Sankyo; Tamaki el al., 1995, Chem. Pharm. Bull. 43:1883-1893).

Where the MMP inhibitor is tetracycline or a tetracycline derivative, the local concentration of inhibitor in the area of skin to be treated is preferably between 0.5 and 50 μg/ml, and preferably between 10 and 20 μg/ml. Where the term “between” is used to indicate a range of values herein, it is intended to include the limits of the range, unless specifically indicated otherwise. The serum or plasma concentration of tetracycline or tetracyline derivative may likewise be between 0.5 and 50 μg/ml, and preferably between 10 and 20 μg/ml. The appropriate concentrations for MMP inhibition by various compounds may be determined using known potency values for those compounds, including but not limited to information contained in publications such as Lokeshwar et al., 1998, Adv. Dentl. Res. 12:97-102; Mäkelä et al., 1998, Adv. Dent. Res. 12:131-135; Golub et al., 1998, Adv. Dent. Res. 12:170-176; Tekoppele et al., 1998, Adv. Dentl. Res. 12:63-67; Vemillo and Rifkin, 1998, Adv. Dentl. Res. 12:56-62; Sefton et al., 1998, Adv. Dentl. Res. 1:103-110; Golub et al., 1998, Adv. Dent. Res. 12:12-26; Ciancio, 1998, Adv. Denti. Res. 12:27-31; and Masumori et al., 1998, Adv. Dentl. Res. 12:111-113.

For bydroxymate and its derivatives, an effective local or serum concentration may be in the range of 100-1500 ng/ml.

For carboxylic acid derivatives, an effective local or serum concentration may be in the range of 100-1500 ng/ml.

For other MMP inhibitors, the effective local concentration may be determined based on (i) the relative potency of the inhibitor in inhibiting MMP compared to a tetracycline, such as doxycycline, and (ii) the effective local concentration of the tetracycline, as set forth above.

In alternative embodiments, one or more than one MMP inhibitor may be administered to a subject at any one time.

MMP inhibitor may be administered by any suitable route, including systemic and/or local administration. For example, where lesions are widespread, or it is desired to prevent the development of new lesions, MMP inhibitor may desirably be administered systemically, such as by oral or intravenous administration. For more localized disease, or where higher concentrations of inhibitor at the lesion site are desired, local administration, for example using a topical formulation or cutaneous or subcutaneous injection or implant, may be more appropriate. The dosages administered in each instance are adjusted to provide the local concentrations of inhibitor set forth above.

For example, where the inhibitor is tetracycline or a tetracycline derivative and is orally administered, the daily dosage for an adult may range from 250 to 2000 mg (5 to 20 mg/lb for children older than eight years, where tetracyclines are contraindicated in children younger than 8 years and in pregnant women). In particular non-limiting embodiments, where the inhibitor is tetracycline, the daily dose may be 250 to 2000 mg (5 to 20 mg/lb for a child older than eight years); where the inhibitor is doxycycline, the daily dose may be 25 to 200 mg (for children older than eight years and under 100 lbs, 0.5 to 1 mg/lb); and where the inhibitor is minocycline, the daily dose may be 25-200 mg (for children older than eight years, 2 to 4 mg/kg). Where the inhibitor is another tetracycline derivative, the dose may be adjusted based on the relative potency of the derivative in inhibiting MMP compared to another tetracycline, such as doxycycline, and other bioavavailability considerations, using techniques known in the art.

The preferred route of administration for a MMP inhibitor depends upon the bioavailability of the compound. MMP inhibitors with good bioavailability may be administered by systemic routes (such as oral, intravenous) to achieve effective local concentrations.

In non-limiting embodiments, where the inhibitor is hydroxymate or a hydroxymate derivative and is orally administered, the daily dosage for an adult may range from 10 to 200 mg, preferably 20 to 150 mg. For other hydroxymate derivatives, the dose may be adjusted based on the relative potency of the hydroxymate derivative in inhibiting MMP compared to a tetracycline, such as doxycycline, and other bioavavailability considerations, using techniques known in the art.

In specific non-limiting embodiments, Marimastat may be administered orally at a daily dosage of 20 to 50 mg ( e.g., 10 to 25 mg administered twice daily; Primrose et al., 1996, Ann. Oncol. 7(Supp.):47; Parsons et al., 199, Ann. N. Y. Acad. Sci. 878:47);, RO 32-3555 may be administered orally at a daily dosage of 10 to 100 mg (Wood et al., 1996, Br. J. Clin. Pharmacil. 42:676-677); and BAY 12-9566 may be administered orally at a daily dosage of 20 to 100 mg (Leff, 1999, Ann. N.Y. Acad. Sci. 878:201-220). The serum levels of these agents may vary from about 50 to 1200 ng/ml, depending upon the interval between dosing and measurement.

Inhibitors with poor bioavailability, such as carboxylic and phosphonic acid derivatives, may preferably be administered topically (e.g. in lotions, creams, ointments, or under occlusion dressings).

In non-limiting embodiments, where the inhibitor is a carboxylic acid derivative, it may be topically administered at a concentration of from 0.1 to 1.0% (where percent is weight percent). For specific carboxylic acid derivatives, the dose may be adjusted based on the relative potency of the carboxylic acid derivative in inhibiting MMP compared to a tetracycline, such as doxycycline, and other bioavavailability considerations, using techniques known in the art.

In other non-limiting embodiments, where the inhibitor is a phosphonic acid derivative, it may be topically administered at a concentration of from 0.1 to 1 percent (where percent is weight percent). For specific phosphonic acid derivatives, the dose may be adjusted based on the relative potency of the phosphonic acid derivative in inhibiting MMP compared to a tetracycline, such as doxycycline, and other bioavavailability considerations, using techniques known in the art.

Where the MMP inhibitor is to be administered locally, for example, as a topical formulation (including but not limited to a lotion, cream, aqueous or alcoholic solution or suspension), the concentration of inhibitor in the formulation may be equal to or greater than the desired local tissue concentration. Preferably, the concentration of MMP inhibitor is desirably 10 to 100 times the IC₅₀ of the MMP inhibitor (where IC₅₀ results in 50% reduction of the MMP enzymatic activity in vitro). Specific nonlimiting examples are topical formulations of hydroxymate, BAY 12-9566 or phosphonate derivatives at concentrations 10-100 fold greater than the IC₅₀ of those compounds, where the IC₅₀ for hydroxymate is about 1-5 mM, the IC₅₀ for BAY 12-9566 is about 2.2-60 μM, and the IC₅₀ for phosphonate derivatives in about 2.5 mM. Further, for topical formulations, it may be desirable to include an agent which facilitates the passage of the MMP inhibitor(s) into the skin. Such agents include but are not limited to dimethylsulfoxide (“DMSO”; e.g., at a concentration between 0.125 and 0.9 percent), retinoic acid, alpha hydroxy acids, and salicylic acid. For any of the modes of administration, a total daily dosage set forth above may be administered as a single dose or in divided doses.

The present invention further provides for topical formulations of tetracycline and tetracycline derivatives, including, but not limited to, natural tetracyclines, such as chlortetracycline, oxytetracycline, and tetracycline; semi-synthetic tetracyclines such as minocycline, doxycycline, and methacycline; and chemically modified tetracyclines (“CMTs”) such as CMT-1 (4-dedimethylamino-tetracycline), CMT-2 (tetracycline-nitrile), CMT-3 (6-demethyl-6-deoxy-4-dedimethylaminotetracycline); CMT-4 (7-chloro-4-dedimethylaminotetracycline); CMT-5 (tetracyclinepyrazole); CMT-6 (4-dedimethylamino-4-hydroxytetracycline); CMT-7 (12-alpha-deoxy-4-dedimethylaminotetracycline); and CMT-8 (6 alpha deoxy-5 hydroxy-4-dedimethylaminotetracycline). The concentrations of these tetracyclines may be between 0.1 and 1.0 percent (where percent is weight percent). Skin penetration of said tetracycline compounds may be enhanced by adding dimethylsulfoxide, for example at a concentration of between 0.125 and 0.9 percent, and/or retinoic acid, and/or an alpha-hydroxy acid, and/or salicylic acid.

The present invention provides for pharmaceutical compositions comprising one or more MMP inhibitor for use in treating psoriasis. Such compositions may further comprise other active or inactive components. Examples of active components include, but are not limited to, corticosteroids and other immunosuppressant compounds, antibiotics, antiinflammatory agents, retinoids, psoralen compounds, etc.

In additional embodiments, the present invention provides for methods for diagnosing psoriasis, or a predisposition toward psoriasis, or a skin disease amenable to treatment by MMP inhibitors, comprising determining that a skin sample of a subject (i) exhibits MMP-2 expression in suprabasal keratinocytes; (ii) exhibits TIMP-2 expression in suprabasal keratinocytes; (iii) exhibits active MMP-2 by zymogen or Western blot tests; and/or (iv) exhibits proMMP-9 expression by Western blot analysis, wherein observation of any of the features (i)(iv) bears a positive correlation with a diagnosis of active or inactive psoriasis. Identifying any one of these features indicates that a patient's skin condition may respond to treatment with an MMP inhibitor.

In still further embodiments, the present invention provides for methods of evaluating the level of disease activity in a psoriasis patient or a person suspected of suffering from psoriasis, comprising measuring the level of MMP-2 and/or TIMP-2 in serum, and comparing the measured level with a control sample, where an increase in the level of either enzyme would have a positive correlation with the level of disease activity (e.g., an increase in the level of enzyme would correlate with an increase in disease activity). The control sample may be obtained from either a healthy subject (who does not suffer from psoriasis) or may be a sample previously obtained from the patient (so as to monitor the course of an individual's disease). The level of MMP-2 or TIMP-2 may be evaluated, for example but not by way of limitation, by ELISA techniques using specific polyclonal or monoclonal antibodies directed toward MMP-2 or TIMP-2. ELISA for MMP-2 is described in Zucker et al., 1992, J. Immunol. Meth. 148:189-198; ELISA for TIMP-2 is described in Fujimoto et al., 1993, Clin. 220:31-45).

In further embodiments, the present invention provides for transgenic animal and cell and tissue culture models for psoriasis comprising a cell or animal genetically engineered to overexpress MMP-2 and/or TIMP-2 in keratinocytes. For example, transgenic animals or isolated keratinocyte cells may be engineered to contain a MMP-2 or TIMP-2 transgene operatively linked to a promoter active in keratinocytes, such as, but not limited to, the keratin 5, keratin 10, keratin 16 or involucrin promoter (Carrol et al., 1995, Cell 83:957-968) or the haptoglobin promoter (D'Armiento et al., 1995, Mol. Cell Biol. 15:5732-5739) In other particular embodiments, keratinocyte expression of a transgene may be induced in keratinocytes by operatively linking an MMP-2 or TIMP-2 encoding nucleic acid to a topically inducible promoter, for example a promoter activated by ultraviolet light or by topical application of an inducer. Suitable inducible promoters would include the mouse metallothionien promoter (Palmiter et al., 1982, Cell 29:701). Keratinocytes engineered to incorporate an inducible MMP-2gene could be incorporated into a skin culture in vitro and thereby create a tissue culture system for studying psoriasis. The skin culture may comprise a portion of natural skin, for example, a natural dermis, or may be completely synthetic. Artificial skin culture systems are example, a natural dermis, or may be completely synthetic. Artificial skin culture systems are known in the art.

Besides their inhibitory effects on MMPs, tetracyclines have also been shown to have additional anti-inflammatory properties that would be beneficial in the treatment of psoriasis. These include:

(1) Overproduction of nitric oxide has been shown to play an important role in inflammatory diseases including arthritis. It has been shown that doxycycline, minocycline and chemically modified tetracyclines inhibit nitric oxide synthetase expression (Amin et al. Proc. Nal Acad Sci, USA 1996;93:14014-9) and (Amin et al. FEBS Lett 1997;410:259-64).

(2) It is known that phospholipase A₂ plays a role in the inflammatory process. In this regard, both minocycline and doxycycline inhibit phospholipase A₂ (Prozanski et al. Biochem Pharmacol 1992;44:1165-70).

(3) Proliferation and activation of T lymphocytes is a major feature in the patho-physiology of psoriasis (Chang et al. Proc Nat Acad Sci, USA 1994;91:9283-86). It has been shown that minocycline in a dose-dependent fashion inhibits T lymphocyte proliferation and reduces the production of IL-2, IFN-δ and TNF-α all of which have been incriminated in psoriasis (Kloppenburg et al. Clin Exp Immuno 1995;102:635-41).

Thus tetracyclines may be beneficial in the treatment of psoriasis as the result of multi-anti-inflammatory effects including (a) inhibition of MMPs, (b) reduction in activation of T lymphocytes, and (c) an inhibitory effect on nitric oxide and phospholipase A₂.

### 6. EXAMPLE

### MMP-2 and its Inhibitor, TIMP-2, are Overexpressed in Psoriatic Epidermis

### 6.1. Materials and Methods

Patients: Seventeen patients with widespread plaque psoriasis were selected for this study. There were 13 males and 4 females, ranging in age from 27 to 73 years old, and duration of disease ranged from 5 to 30 years. Eight patients were in treatment with UVB, 2 with PUVA , and 7 were on no treatment or were only using topical steroids. None of the patients were on methotrexate or retinoids. Skin biopsies under local xylocaine anesthesia were performed from distal uninvolved and involved skin. All patients were provided with informed written consent forms previously approved by the Institutional Review Board at the Mount Sinai Medical Center in New York City. Normal control skin from non-psoriatic patients was obtained from post-surgical specimens.

Source of Antibodies: Antibodies were generous gifts or purchased as stated below. Antibodies against basement membrane components were as follows: affinity purified rabbit polyclonal anticollagen IV (H. Kleinmann, National Institutes of Health, Bethesda, Md.; mAb against the α1 (IV) and α2 (IV) collagen chains (Y. Ninomiya, University Medical School, Okayama, Japan; Ninomiya et al., 1995, J. Cell Biol. 130:1219-1229); laminin anti-α2 chain, mAb 5H2-F7 (E. S. Engvall, La Jolla Cancer Research Foundation, Calif.; Engvall et al., 1990, Cell Regulation 1:731-740); laminin anti-α5 chain, mAb 4C7 (GIBCO, Grand Island, N.Y.); laminin anti-β1 chain, mAb 1921 (Chemicon International, Temecula, Calif.; Engvall et al., 1986, J. Cell Biol. 103:2457-2465); laminin anti-γ1 chain, mAb D-18 (Developmental Studies Hybridoma Bank, University of Iowa; Sanes et al., 1990, J. Cell Biol. 111:1685-1699); EHS-laminin rabbit polyclonal antibody (H. K. Kleinmann, National Institutes of Health, Bethesda, Md. Antibodies against metalloproteinases and inhibitors were as follows: mAb#1346 (anti-human MMP-1); mAb#3308 (anti-human MMP-2); mAb#3309 (anti-human MMP-9), and mAb#3319 (anti-MT1-MMP) (Chemicon International, Temecula, Calif.; Fujimoto et al., 1993, Clin Chim Acta 221:91-103); rabbit polyclonal Ab-45 (anti-human MMP-2 ; W. G. Stetler-Stevenson, National Institutes of Health, Bethesda, Md.; Monteagudo et al., 1990, Am. J. Pathol. 136:585-592); rabbit polyclonal Ab-485 (anti-human TIMP-2; H. Birkedal-Hansen, National Institutes of Health, Bethesda, Md.; mAb#3310 (anti-TIMP-2; Chemicon International, Temecula, Calif.; Fujimoto et al., 1995, J. Immunol. Methods 187:33-39).

Immunochemistry: Skin specimens were frozen in Tissue-Tech OCT embedding compound (Miles Labs, Elkhart, Ind.). Indirect immunofluorescence microscopy was performed as described in Fleischmajer et al., 1993, J. Histochem. Cytochem. 41:1359-1366. Specimens were examined using a microscope equipped with epiflourescence illumination or by confocal laser scanning microscopy. Controls consisted of pure mouse IgG or rabbit or mouse serum from non-immunized animals. In some specimens nuclei were visualized with propidium iodide staining.

Electron Microscopy: Skin specimens were immediately immersed in a fixative solution containing 3% glutaraldehyde with 0.2 M sodium cacodylate at pH 7.4. After overnight fixation the fixative solution was removed and replaced with a phosphate buffer followed by 1 percent osmium tetroxide buffered with sodium cacodylate. After one hour the osmium was replaced with increasing concentrations of ethanol through propylene oxide and into embed 812. One micrometer plastic sections were cut, stained with methyl blue and azure II and observed by light microscopy. Representative areas were chosen for ultrathin sectioning and observed with a JEM 100CX transmission electron microscope (JEOL, LTD, Tokyo, Japan).

Substrate Gel Electrophoresis (Zymography): Metalloproteinases were detected and characterized by zymography (Nakajima et al., 1995, Br. J. Cancer 71:1039-1045). Uninvolved and involved skin specimens from 4 patients and 2 normal controls were extracted in 100 mM Tris-HCL pH 8.0 and 0.1 percent Triton X-100. Thirty micrograms of Triton soluble protein as determined by the B.A. method (Pierce, Rockford, Ill. ) were loaded on 8 percent SDS-PAGE gels that had been co-polymerized with 1 mg/ml gelatin. Electrophoresis was performed under non-reducing conditions at 100 volts for 2 hr at 4° C. Gels were washed once for 30 minutes in 2.5 percent Triton X-100 to remove SDS and were then incubated in collagenase buffer (100 mM Tris-HCL pH 8.0, 5 mM CaCl2, 0.02 percent NaN₃), for 40 h at 37° C. Gels were stained with 0.5 percent Coomassie blue in 30 percent methanol/10 percent acetic acid for 30 minutes at room temperature and de-stained in 30 percent methanol/10 percent acetic acid three times for 15 minutes. The presence of metalloproteinases was indicated by an unstained proteolytic zone in the substrate. Fibroblast-conditioned medium, obtained by a 24-hour incubation of NIH-3T3 cells with 50 μg/ml ascorbic acid in serum-free DMEM media, was used as a positive control for MMP-2.

Western Immunoblots: Tissues were extracted with a lysis buffer (50 mM Tris-HCl, pH 7.5, 150 mM NaCl, 1 percent N P-40 and 5 mM EDTA). Tissue extracts (5 μg protein per lane) were run in SDS-polyacrylamide gels (10 percent for MMP-2 and MT1-MMP and 15 percent for TIMP-2) and transferred to polyvinylidene fluoride membranes. MMP-2 was detected by incubation with mAb#3308 or by rabbit polyclonal Ab-45, while TIMP-2 was detected with mAb #3310 or with rabbit polyclonal antibody Ab-485 and MT1-MMP was detected with mAb#3319. The runs were visualized by an enhanced chemi-fluorescence detection system (Amersham Pharmacia Biotech Inc., Piscataway, N.J.).

In situ Hybridization: A human MMP-2, 3kb cDNA and a human MMP-9, 2.4 kb cDNA were subcloned into the vector ECO R1. Riboprobes were generated by using T3 (antisense) and T17(sense) RNA polymerase. (Gift of G. Stetler-Stevenson, National Institutes of Health, Bethesda, Md.). The digoxigenin-labeled sense and antisense riboprobes were prepared by in vitro transcription using a RNA labeling kit (Boelringer Mannheim, Indianapolis, Ind.). Tissues were fixed in 4 percent paraformaldehyde in PBS (pH 7.2) for 24-48 hours, dehydrated, embedded in paraffin, and serially sectioned at 5 μm. Sections were placed on triethoxysilane-treated slides, dried overnight at 37° C., and stored at 4° C. until used. After deparaffinization, the sections were treated with proteinase K (10 μg/ml) for 15 minutes followed by 0.2N HCl for 5 minutes at room temperature. After washings in PBS, sections were acetylated to make mRNA more available for hybridization. Hybridization was performed in hybridization solution containing 50 percent formamide, 10 mM Tris-HCl, pH 7.6, 200 μg/ml tRNA, 1×Denhardt's solution, 10 percent dextran sulfate, 600 mM NaCl, 0.25 percent SDS, and 1 mM EDTA at 50° C. for 16 hours in a humidified chamber. Slides were washed in 2×SSC containing 50 percent formamide and non-hybridized transcripts were digested with 20 μg/ml RNase A (Nieto et al., 1996, Methods Cell Biol. 51:219-235). Detection of the hybridized cRNA was carried out using the Genius Detection System (Boehringer Mannheim, Indianapolis, Ind.), in which the specific transcripts were detected with anti-digoxigenin antibody conjugated to alkaline phosphatase. Finally, the slides were immersed in the color-development solution (0.3 mg/ml Nitro Blue Tetrazolium and 0.15 mg/ml 5-bromo-4-chloro-3-indolyl phosphate in 0.1 M NaHCO₃). The slides were examined using a bright field microscope.

### 6.2. Results

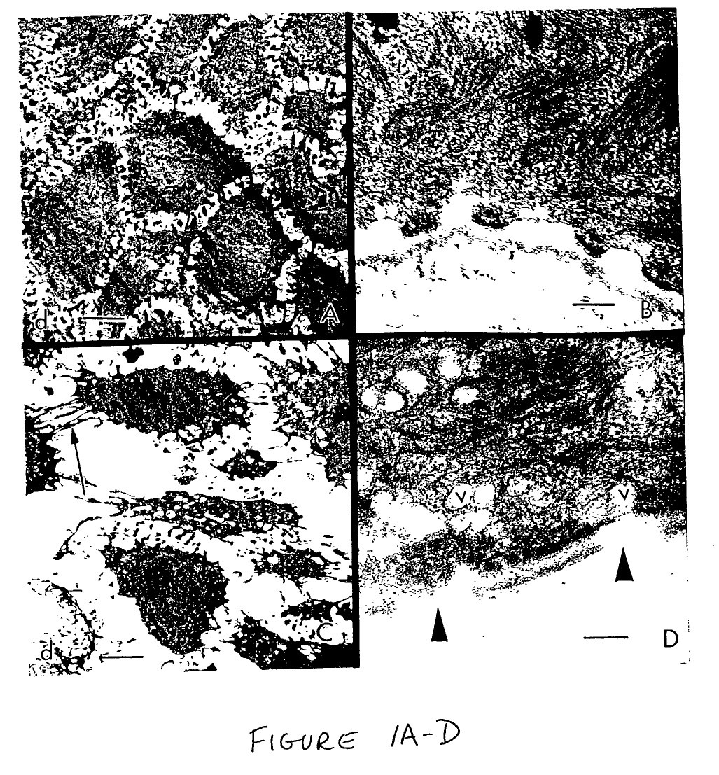

Electron Microscopy: Transmission electron microscopy was performed on uninvolved and involved skin from 2 psoriasis patients and a normal control. Most of the alterations were noted in the involved psoriatic skin. There was a marked increase in intercellular spacing between keratinocytes in the basal and suprabasal layers accompanied by a reduction of intercellular bridges, which appeared thin and elongated (FIG. 1C). Desmosomes were reduced in numbers and were often found loose in the intercellular spaces. Basal keratinocytes showed numerous coated vesicles, some opening into the lamina lucida (FIG. 1D). The lamina densa revealed gaps and in some areas splitting or reduplication (FIG. 1D). The above changes suggested alterations in cell-cell and cell-matrix adhesion in involved psoriatic skin.

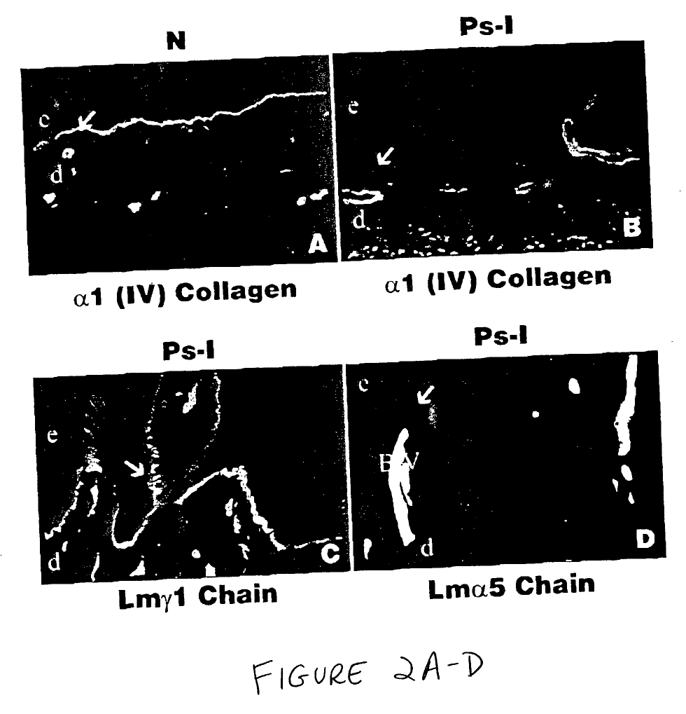

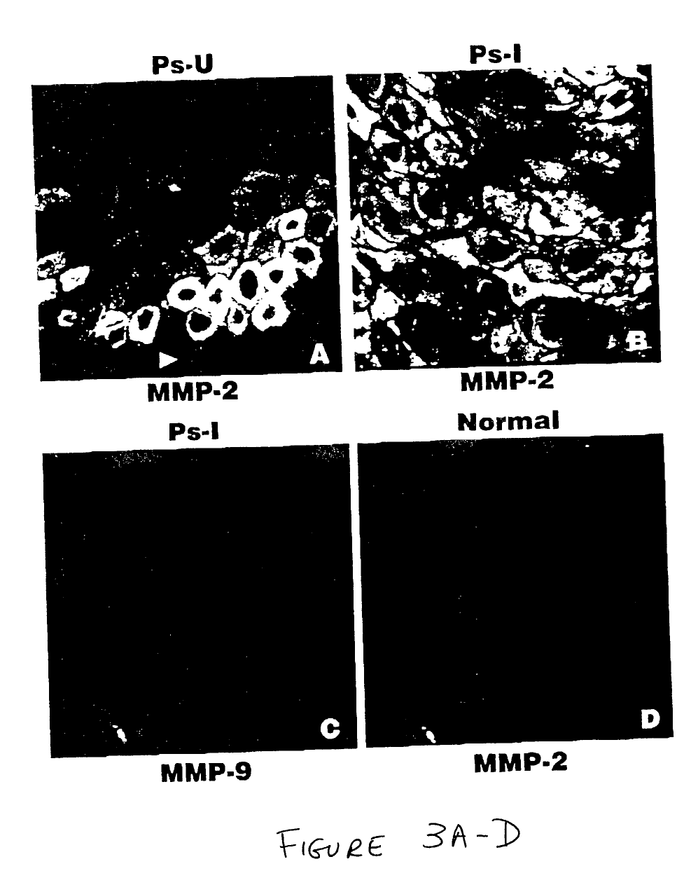

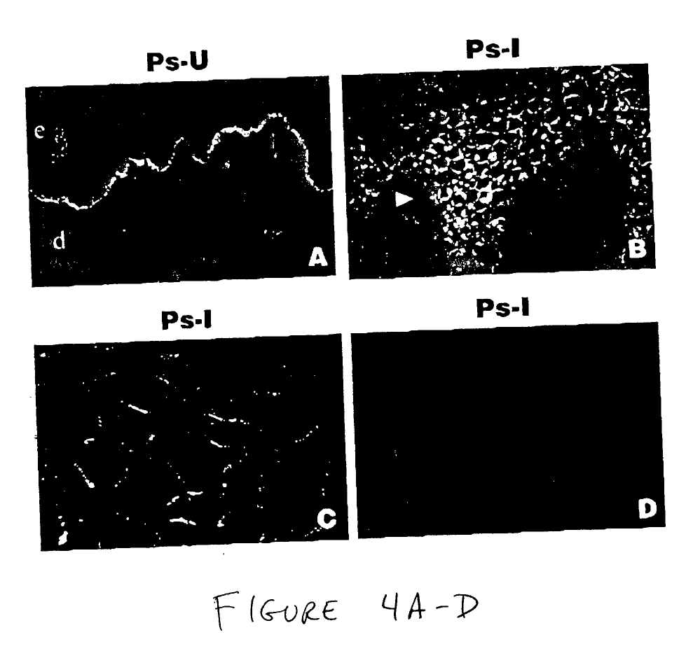

Immunochemistry of Collagen IV and Laminins: Basement membranes were studied by immunohistochemistry in 7 patients (uninvolved and involved psoriatic areas) and 4 normal controls. Indirect immunofluorescence microscopy was performed with antibodies against α1 (IV), α2 (IV) collagen chains and against various laminin chains α2, α5, β₁, γ₁). All psoriatic specimens showed alterations of the basement membrane at the epidermal-dermal interphase, although the changes were more pronounced in the involved areas. Three abnormalities were noted in the basement membrane at the epidermal-dermal interphase, namely (i) large gaps or areas with reduced staining intensity, (ii) areas of splitting of the basement membrane into several layers, and (iii) marked folding (FIG. 2). These changes were more pronounced at the tip of the elongated rete ridges. Superficial capillaries were increased in number and showed dilation or tortuosity, but their basement membrane stained normal with collagen IV and laminin antibodies. Immunohistochemistry of MMP-2 and TIMP-2: Uninvolved and involved skin from 8 patients with psoriasis and 4 normal controls were studied by immunohistochemistry with antibodies against MMP-2, TIMP-2, MMP-9, MMP-1 and MT1-MMP. Since ultraviolet light may affect the expression of skin MMPs (Fisher et al., 1996, Nature 379:335-339), 3 patients included in this series were never previously treated with UVL, 3 received UVB and 2 were on PUVA. Since there were no differences between the groups, the results will be presented together. MMP-2 was not detected in uninvolved psoriatic skin in 7 patients. However, 1 patient revealed MMP-2 in the cytoplasm of suprabasal keratinocytes in the rete ridges but not in those localized in the suprapapillary areas. It is noteworthy that suprabasal keratinocytes in involved areas were markedly increased in size when compared to those of uninvolved areas (FIGS. 3A, 3B). Epidermal expression of MMP-2 was strong in 4 patients and less pronounced in the other 4 patients. The dermis did not stain for MMP-2 in psoriatic skin and MMP-9 and MMP-1 were not detected (FIG. 3C). Normal, control skin was negative for MMP-1, MMP-2 and MMP-9 (FIG. 3D). TIMP-2 was present in uninvolved and involved psoriatic skin but it showed two distinct patterns of distribution. Uninvolved psoriatic skin revealed TIMP-2 at the cell surface of basal keratinocytes facing the ECM (FIG. 4A). A similar, but less intense staining was noted in the normal controls. All psoriatic involved specimens revealed TIMP-2 at the cell surface of suprabasal keratinocytes in a linear pattern (FIG. 4B). High magnification, by confocal laser microscopy, revealed a distinct, dot-like, linear pattern suggesting that TIMP-2 was bound to a cell surface receptor (FIG. 4C). Since there is evidence that TIMP-2 binds at the cell surface to MT1-MMP (Sato et al., 1994, Nature 370:61-65, Strongin et al., 1995, J. Biol. Chem. 270:5331-5338; Will et al., 1996, J. Biol. Chem. 271:17119-17123), additional staining was performed with a mAb against MT1-MMP. The results were negative, suggesting that the epitopes for MT1-MMP were cryptic in the epidermis since MT1-MMP was clearly demonstrated by Western blots (see below).

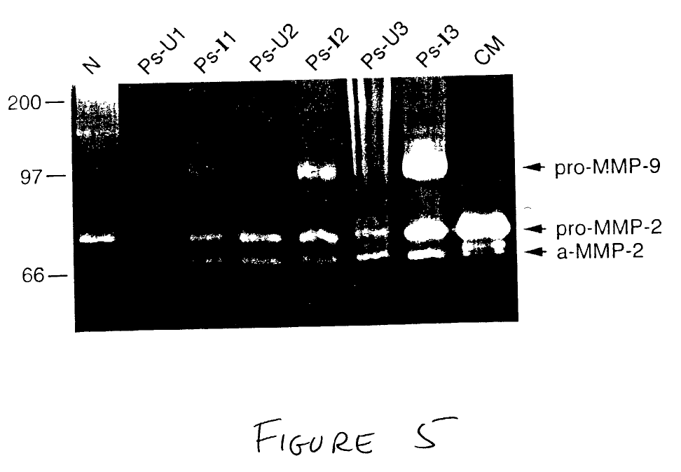

Zymography of MMP-2 and TIMP-2: Extracts of uninvolved and involved psoriatic skin from 4 patients and 2 normal controls were studied by gelatin zymography to determine the presence of pro-MMP-2 and pro-MMP-9, as well as their activated forms. Uninvolved and involved psoriatic skin revealed both pro-MMP-2 and a-MMP-2. On the other hand, pro-MMP-9 was only noted in involved skin. (FIG. 5) The presence of proMMP-9 may be due to local synthesis by keratinocytes or may have been transferred to the epidermis by neutrophils, which are known to store MMP-9 (Birkedal-Hansen, 1995, Curr. Opin. Cell Biol. 7:728-735). Normal control skin expressed mostly pro-MMP-2.

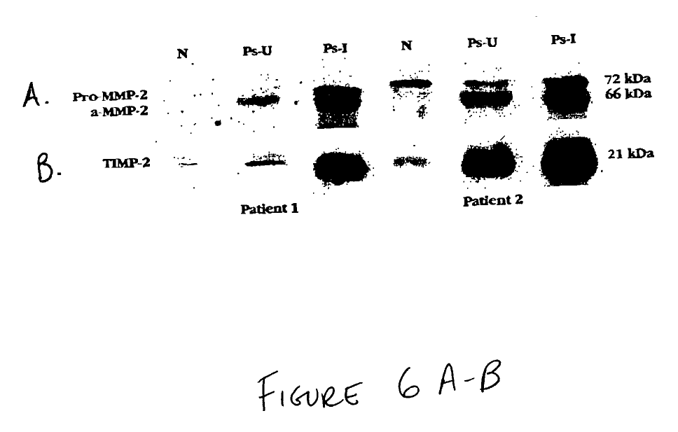

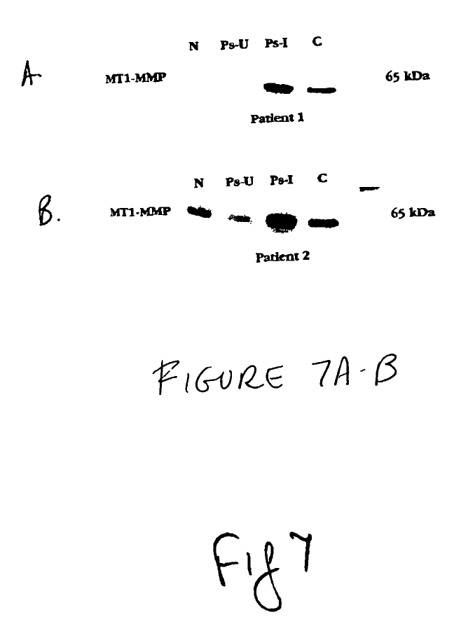

Western Blots of MMP-2 and TIMP-2: Western blots with monoclonal and polyclonal antibodies were performed on extracts of skin samples from the above 4 patients. Pro-MMP-2 and a-MMP-2 were expressed in both uninvolved and involved psoriatic skin, although the signal was more intense in the latter (FIG. 6). Normal control skin was negative or only expressed pro-MMP-2. (FIG. 6) TIMP-2 was strongly expressed in uninvolved and involved psoriatic skin, although the signal was more intense in the latter (FIG. 6). Normal control skin showed mild expression of TIMP-2. MT1-MMP was also increased in involved psoriatic skin, as shown by Western blots. (FIG. 7).

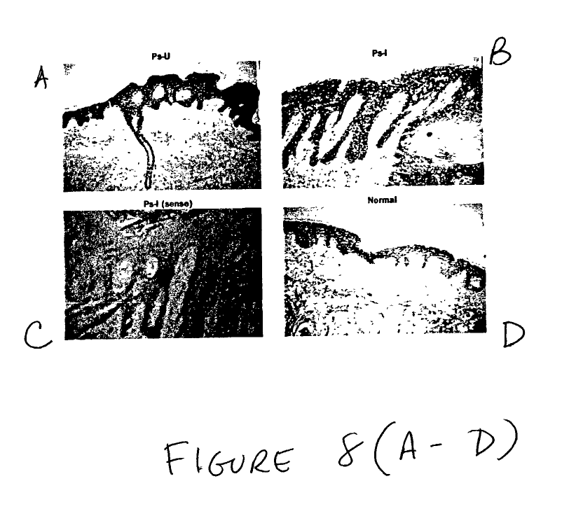

MMP-2 mRNA expression by in situ hybridization: Specimens from 2 patients (uninvolved and involved skin) and from a normal control skin were studied for expression of MMP-2 mRNA by in situ hybridization. Psoriatic uninvolved and involved skin showed strong cytoplasmic signals in the suprabasal layers, as well as in ecrine sweat ducts (FIG. 8). Hybridizations with sense MMP-2 mRNAs were essentially negative. Weak signals for MMP-9 mRNA were also noted in involved psoriatic skin. Normal control skin was basically negative for both MMP-2 and MMP-9 mRNAs. A full length human TIMP-2 cDNA was subcloned into ECO R1, and riboprobes generated for in vitro transcription. Data showed TIMP-2 mRNA signals in involved psoriatic skin.

### 6.3. Discussion

The foregoing study showed suprabasal expression of MMP-2 and TIMP-2 in uninvolved and involved psoriatic skin. The increase in MMP-2 protein and MMP-2-mRNA in uninvolved skin supports the concept that psoriasis is a body wide disease. Early studies involving transplantation of uninvolved and involved psoriatic skin into athymic mice showed that both were affected, as determined by measuring cell proliferation and plasminogen activator levels (Krueger et al., 1981, J. Clin. Invest. 68:1548-1557; Fraki et al., 1982, Science 115:685-687). The overexpression of MMP-2 in psoriatic skin raises the question as to whether such an increase may be the result of cytokine stimulation by inflammatory cells. This hypothesis appears unlikely since strong signals for MMP-2-mRNA were also noted in uninvolved skin where inflammatory cells are rarely present. Furthermore, it is known that MMP-2 responds poorly to cytokine stimulation, with the exception of TGF-,β1 (Salo et al., 1991, J. Biol. Chem. 266:11436-11441; Overall et al., 1991, J. Biol. Chem. 266:14064-14071). It has been shown that fibroblasts and keratinocytes can be moderately stimulated to produce MMP-2 by TGF-β1. However, the presence of TGF-β1 in psoriasis is controversial since although an increase was shown by immunochemistry (Kane et al., 1990, J. Cell Physiol. 144:114-150), no expression of MMP-2-mRNA could be demonstrated by in situ hybridization (Shmid et al., 1993, Arch Dermatol. Res. 285:334-340). Furthermore, it has recently been shown that TGF-β1 stimulation has no effect on MMP-2-mRNA levels but it increases the stability of the secreted pro-enzyme (Sehgal et al., 1999, Mol. Biol. Cell 10:407-416).

MMP-2 is expressed in the skin during wound healing (Salo et al., 1994, Lab Invest 70:176-182) and in certain tumors (Pyke et al., 1992, Cancer Res. 52:1336-1341) but usually in the dermis, where it plays a role in the remodeling of the ECM. However, MMP-2 may have additional biological roles involving cell proliferation, adhesion, and migration (Yu et al., 1998, In “Matrix Metalloproteinases”. Parks WC, Mecham RP (Eds). Academic Press pp. 85-113). The role of MMP-2 in psoriatic epidermis remains unknown although it is noteworthy that cell proliferation and angiogenesis, prominent features in psoriasis, are reduced in MMP-2 deficient mice (Itoh et al., 1998, Cancer Res. 58:1048-1051). It has also been shown that ectopic epidermal, suprabasal expression of MMP-1 or collagenase-1 in transgenic mice results in acanthosis, hyperkeratosis, and disruption of intercellular contacts, suggesting a psoriatic phenotype (D'Armiento et al., 1995, Mol. Cell Biol. 15:5732-5739). This raises the question as to whether intercellular disruption of suprabasal keratinocytes in psoriasis may be due to activation of pro-MMP-2.

The suprabasal expression of TIMP-2 at the cell surface of psoriatic keratinocytes is of considerable interest. TIMP-2 is constitutively expressed in mice skin during embryogenesis and adult life (Blavier et al., 1997, Mol. Biol. Cell 8:1513-1527). However, in situ hybridization studies showed its mRNA in the dermis and around hair follicles but not in the epidermis (Blavier et al., 1997, Mol. Biol. Cell 8:1513-1527). TIMP-2-mRNA was also noted in the stroma of basal cell and epidermoid carcinomas of the skin (Wagner et al., 1996, J. Invest. Dermatol. 106:321-326). It is known that TIMP-2, besides acting as a specific inhibitor for MMP-2, has other biological functions involving regulation of cell proliferation and survival (Gomez et al., 1997, Eur. J. Cell. Biol. 74:111-122, Blavier et al., 1999, Ann. N.Y. Acad. Sci. 878:108-119). The presence of TIMP-2 at the cell membrane of suprabasal keratinocytes is most unusual and raises questions about its role in the activation of pro-MMP-2 as well as its role on cell proliferation. It is known that TIMP-2 has great affinity for cell surfaces and forms a bridge between MT1-MMP and pro-MMP-2 (Goldberg et al., 1989, Proc. Natl. Acad. Sci. (U.S.A.) 86:8207-8211, Sato et al., 1994, Nature 370:61-65, Strongin et al., 1995, J. Biol. Chem. 270:5331-5338; Will et al., 1996, J. Biol. Chem. 271:17119-17123). The mechanism of pro-MMP-2 activation depends on the tissue levels of TIMP-2 and MT1-MMP. Thus, high concentrations of TIMP-2 have an inhibitory effect on pro-MMP-2 while low levels allow MT1-MMP to activate pro-MMP-2 (Strongin et al., 1995, J. Biol. Chem. 270:5331-5338; Will et al., 1996, J. Biol. Chem. 271:17119-17123). The role of TIMP-2 on cell proliferation has been shown to be paradoxical. Although TIMP-2 inhibits the growth of human melanoma cells (Montgomery et al., 1994, Cancer Res. 54:5467-5473), it can stimulate proliferation of a variety of normal and neoplastic cells (Stetler-Stevenson et al. 1992, FEBS Lett. 296:231-234; Nemeth et al., 1993, Exp. Cell Res. 207:376-382; Hayakawa et al., J. Cell Sci. 107:2373-2379). Although the mechanism by which TIMP-2 activates cell proliferation is not well understood, there is evidence that it stimulates adenylate cyclase and cAMP-dependent activation of protein kinase A (Corcoran et al., 1995, J. Biol. Chem. 270:13453-13459).

The overexpression of MMP-2 and TIMP-2 in psoriatic skin is of considerable interest not only as it pertains to the pathogenesis of this disease but also about their biological functions in normal skin. The genes of MMP-2 and TIMP-2 have features of housekeeping genes and they are constitutively expressed in many cells during embryogenesis and adult life (Overall et al., 1991, J. Biol. Chem. 266:14064-14071, Blavier et al., 1999, Ann. N.Y. Acad. Sci. 878:108-119, Salo et al., 1994, Lab Invest 70:176-182, Hammani et al., 1996, J. Biol. Chem. 271:25498-25505). The alteration of cell-cell and cell-matrix adhesion in psoriatic epidermis, in association with an increase in the MMP-2-TIMP-2 complex suggests that these compounds may regulate proteolysis of adhesion molecules and this may control keratinocyte migration and proliferation. Preliminary data showed disruption of E-cadherin; β-catenin and desmoglein expression in psoriatic epidermis, suggesting that desmosomes and adherent junctions may be altered in psoriatic epidermis.

Various publications are cited herein, the contents of which are hereby incorporated by reference in their entireties.

What is claimed is:

1. A method of treating psoriasis comprising administering, to a subject in need of such treatment, a therapeutically effective amount of an inhibitor of a matrix metalloproteinase enzyme.

2. The method of claim 1 where the inhibitor is selected from the group consisting of tetracycline and a tetracycline derivative.

3. The method of claim 1 where the inhibitor is selected from the group consisting of hydroxymate and a hydroxymate derivative.

4. The method of claim 1 where the inhibitor is a carboxylic acid derivative.

5. The method of claim 1 where the inhibitor is a phosphonic acid derivative.

6. The method of claim 1 where the inhibitor is systemically administered.

7. The method of claim 1 where the inhibitor is locally administered.

8. The method of claim 7 where the inhibitor is administered as a topical formulation.

9. The method of claim 2 where the inhibitor is systemically administered.

10. The method of claim 2 where the inhibitor is locally administered.

11. The method of claim 10 where the inhibitor is administered as a topical formulation.

12. The method of claim 3 where the inhibitor is systemically administered.

13. The method of claim 3 where the inhibitor is locally administered.

14. The method of claim 13 where the inhibitor is administered as a topical formulation.

15. The method of claim 4 where the inhibitor is systemically administered.

16. The method of claim 4 where the inhibitor is locally administered.

17. The method of claim 16 where the inhibitor is administered as a topical formulation.

18. The method of claim 5 where the inhibitor is systemically administered.

19. The method of claim 5 where the inhibitor is locally administered.

20. The method of claim 19 where the inhibitor is administered as a topical formulation.

21. A method of diagnosing a MMP inhibitor treatable skin condition in a subject, comprising determining that a skin sample of a subject exhibits a feature selected from the group consisting of (i) MMP-2 expression in suprabasal keratinocytes; (ii) TIMP-2 expression in suprabasal keratinocytes; (iii) active MMP-2 by zymogen testing; (iv) active MMP-2 by Western blot analysis and (v) pro-MMP-9 expression by Western blot analysis; wherein observation of any of the features (i)-(v) bears a positive correlation with responsiveness of the skin condition to MMP inhibitor therapy.

22. A model system for psoriasis comprising keratinocytes genetically engineered to be capable of expressing increased levels of one or more enzyme selected from the group consisting of MMP-2, TIMP-2, and both MMP-2 and TIMP-2.

23. A method of evaluating the level of disease activity in a psoriasis patient, comprising measuring the level of MMP-2 and/or TIMP-2 in serum, and comparing the measured level with a control sample, where an increase in the level of either enzyme would have a positive correlation with the level of disease activity.

24. The method of claim 23 where the control sample is obtained from a subject who does not suffer from psoriasis.

25. The method of claim 23 where the control sample was obtained from the patient on a previous occasion.
